(19) United States
(12) Patent Application Publication
(10) Pub. No.: US 2008/0200893 A1
(43) Pub. Date: Aug. 21, 2008

(54) Sanitary napkin with far-infrared radiation

(76) Inventor: Hsin-Mao Hsieh, Pingtung City (TW)

Correspondence Address: HARNESS, DICKEY & PIERCE, P.L.C., P.O. BOX 828, BLOOMFIELD HILLS, MI, 48303, US

(21) Appl. No.: 11/708,168

(22) Filed: Feb. 15, 2007

(51) Int. Cl. A61F 13/15

(52) U.S. Cl. 604/385.01

(57) ABSTRACT

A sanitary napkin with far-infrared radiation has a napkin body having a plurality of ceramic powders disposed in the napkin body. The plurality of ceramic powders is made of material that is capable of inherently radiating far-infrared rays that can penetrate deeply to human body and resonate with cells to activate cells and accelerate metabolism. Further, the far-infrared rays also have proven utilities of deodorization and bacteriostasis. So, activity of bacteria propagating in menstrual blood can be effectively inhibited and bad smell scented from menstrual blood can be effectively mitigated by the far-infrared rays radiating from the plurality of ceramic powders.

### BACKGROUND OF THE INVENTION

1. Field of the Invention

The present invention relates to a sanitary napkin, and more particularly to a sanitary napkin with far-infrared radiation which is capable of inhibiting propagation of bacteria, deodorizing bad smell and improving a user's physiological condition.

2. Description of Related Art

Sanitary napkins are necessaries for women during menstrual period, which are used to absorb menstrual blood to keep a user's private part dry to avoid discomfort resulted from spilling menstrual blood. However, the sanitary napkin closely contacts with the user's skin to form a warm and humid environment during use, and more particularly becomes a breeding ground for bacteria to incur bacterial infection to the sensitive and fragile skin on the user's private part. Additionally, the environment between the sanitary napkin and the user's skin has poor ventilation, such that menstrual blood collected on the sanitary napkin is easy to scent of bad smell to cause olfactory discomfort to self and other people.

To inhibit propagation of bacteria, a conventional manner is to add bacteriostat to the sanitary napkin. However, the bacteriostat also inhibits propagation of acid probiotic that normally exists in vagina, so that the PH. value of the vagina is unbalanced and may further induce other ailments.

To eliminate or mitigate the bad smell, a conventional manner is to add aromatic to the sanitary napkin. However, the aromatic may become allergens for some users and make skin inflamed or itchy.

### SUMMARY OF THE INVENTION

The primary objective of the present invention is to provide a sanitary napkin with far-infrared radiation to mitigate or obviate the aforementioned problems.

The sanitary napkin with far-infrared radiation has a napkin body. The napkin body has a top, a bottom and a plurality of ceramic powders and may include an absorbent layer for absorbing menstrual blood, a soft contact layer mounted on the top of the absorbent layer for contacting with a user's skin and a impermeable sheet mounted on the bottom of the absorbent layer to prevent menstrual blood from permeating through or leaking out. The plurality of ceramic powders is disposed in the napkin body and could be disposed in the contact layer.

The plurality of ceramic powders is preferably made of aluminum oxide, magnesium oxide, zirconium oxide, titanium dioxide and silicon dioxide in proper proportion and is capable of inherently radiating far-infrared rays that have wavelengths within 4 to 14 micrometers. Frequency of the far-infrared rays is approximately to natural frequency of cell molecules of human body such that the far-infrared rays can penetrate deeply to human body and resonate with cells to activate cells and accelerate metabolism. Further, some experiments have proven that the far-infrared rays also have utilities of deodorization and bacteriostasis.

Thus, activity of bacteria propagating in menstrual blood can be inhibited and bad smell scented from menstrual blood can be mitigated or eliminated by the far-infrared rays radiating from the plurality of ceramic powders to provide a sanitary environment between the napkin body and human body and make a wearer easeful.

Other objectives, advantages and novel features of the invention will become more apparent from the following detailed description when taken in conjunction with the accompanying drawings.

### BRIEF DESCRIPTION OF THE DRAWINGS

FIG. 1A is a cross-sectional side view of a first embodiment of a sanitary napkin with far-infrared radiation in accordance with the present invention;

FIG. 1B is an enlarged cross-sectional side view of the sanitary napkin in FIG. 1;

FIG. 2 is a cross-sectional side view of a second embodiment of the sanitary napkin with far-infrared radiation in accordance with the present invention;

FIG. 3 is a perspective view of the sanitary napkin in FIG. 2; and

FIG. 4 a cross-sectional side view of a third embodiment of the sanitary napkin with far-infrared radiation in accordance with the present invention;

FIG. 5 is a perspective view of the sanitary napkin in FIG. 4.

### DETAILED DESCRIPTION OF THE PREFERRED EMBODIMENT

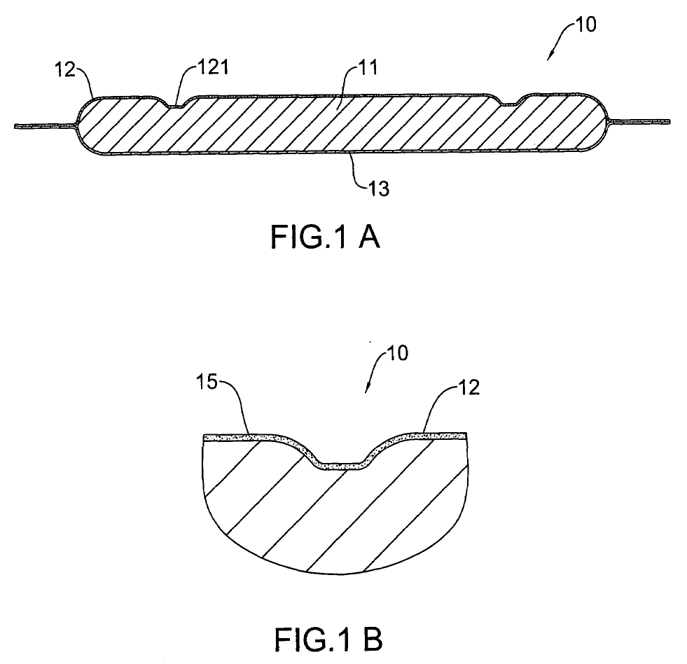

With reference to FIGS. 1A, 1B, 3 and 5, a sanitary napkin with far-infrared radiation in accordance with the present invention comprises a napkin body (10, 10a, 10b).

The napkin body (10, 10a, 10b) has a plurality of ceramic powders (15) and may be implemented in several ways.

In a preferred embodiment of the napkin body (10, 10a, 10b), the napkin body (10, 10a, 10b) includes an absorbent layer (11), a contact layer (12), an impermeable sheet (13) and an optional surface fiber layer (14, 14b).

The absorbent layer (11) is made of material with high absorptivity of water, e.g. cotton, non-woven cloth, paper pulp or the like, to efficiently absorb menstrual blood. The absorbent layer (11) has a top, a bottom and an optional anti-leaking groove (121). The anti-leaking groove (121) is looped and is formed in the top of the absorbent layer (11) to divide the top of the absorbent layer (11) into a central section and an outer section around the central section. The contact layer (12) is mounted on the top of the absorbent layer (11) and is made of soft material, e.g. PE (Polyethylene), cotton or the like, to contact with a user's skin. The contact layer (12) has a top.

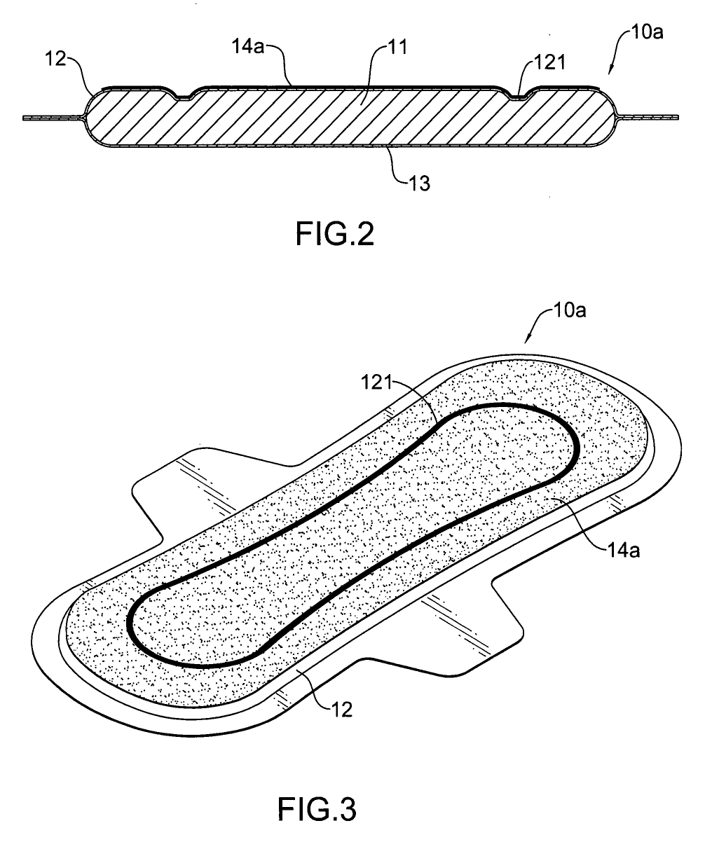

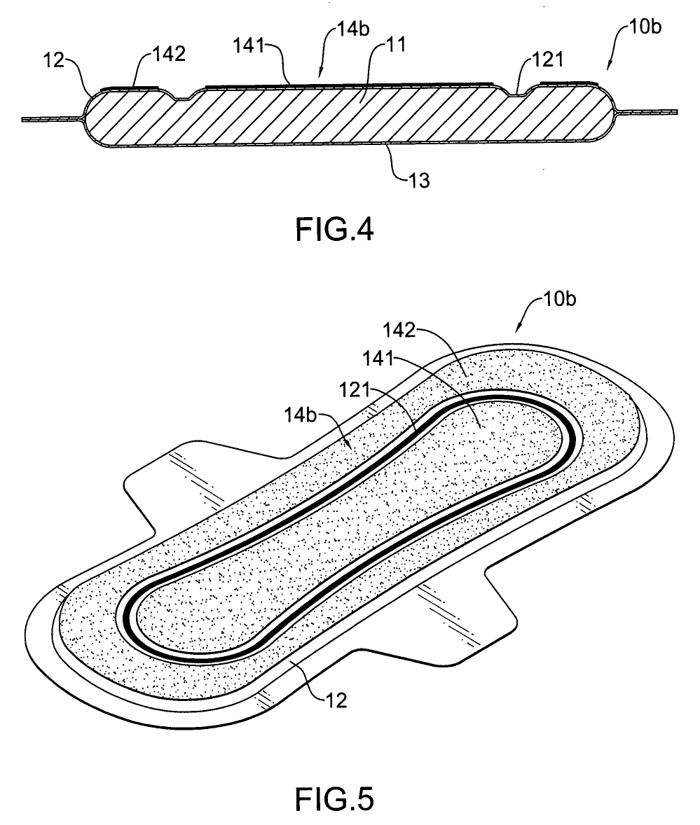

With further reference to FIGS. 2-5, the impermeable sheet (13) is mounted on the bottom of the absorbent layer (11) and is made of waterproof material to prevent menstrual blood from permeating through or leaking out. The surface fiber layer (14a, 14b) is laid on the top of the contact layer (12) and may be an integral piece or multiple separate components. In a preferred embodiment of the surface fiber layer (14b), the surface fiber layer (14b) includes two separate parts respectively being a central component (141) and an outer component (142). The central component (141) is laid on the central section on the absorbent layer (11). The outer component (142) is laid on the outer section on the absorbent layer (11) around the central component (141).

The plurality of ceramic powders (15) is disposed in the napkin body (10, 10a, 10b), may be nanometer-sized and may be implemented in several ways depending on different manufacture method for the napkin body (10, 10a, 10b). The plurality of ceramic powders (15) is preferably made of aluminum oxide, magnesium oxide, zirconium oxide, titanium dioxide and silicon dioxide in proper proportion. Some studies has proven that some kinds of ores mainly containing aluminum oxide, magnesium oxide, zirconium oxide, titanium dioxide and silicon dioxide in specific proportion are capable of radiating far-infrared rays in normal condition and have more radioactive energy in high temperature. Accordingly, the plurality of ceramic powders (15) having composition similar to the aforementioned ores is able to radiate far-infrared rays when being heated.

The far-infrared rays have wavelengths within the range from 4 to 14 micrometers and have been proven, in medical field, that frequency of the far-infrared rays is approximate to natural frequency of cell molecules of human body, so the far-infrared rays have high penetrability to penetrate into human body and resonate with cells to activate cells and accelerate metabolism. Additionally, the far-infrared rays also have utilities of deodorization and bacteriostasis that have been experimentally proven.

Thus, when the napkin body (10, 10a, 10b) contacts with the user's skin to be warmed up, the plurality of ceramic powders (15) continuously radiates far-infrared rays to achieve a far-infrared effect that is inhibiting propagation of bacteria, deodorizing bad smell scented from menstrual blood and improving a user's physiological condition. Additionally, the far-infrared rays penetrate into human body to activate cells and accelerate metabolism such that the user's dysmenorrhea can be significantly eased.

In a first embodiment of the plurality of ceramic powders (15), the plurality of ceramic powders (15) is disposed in the contact layer (12) and may be doped into the contact layer (12) during the manufacture of contact layer (12) or may be mixed with adhesive to be applied on the contact layer (12).

With further reference to FIGS. 2 and 3, in a second embodiment of the plurality of ceramic powders (15), the plurality of ceramic powders (15) is disposed in the surface fiber layer (14a, 14b) which may be an integral piece or multiple separate components. The plurality of ceramic powders (15) may be doped into the surface fiber layer (14a, 14b) during the manufacture of surface fiber layer (14a, 14b) or may be mixed with adhesive to be applied on the surface fiber layer (14a, 14b).

The plurality of ceramic powders (15) disposed in the contact layer (12) or surface fiber layer (14a, 14b) is easier to be warmed up by human body to achieve more efficient far-infrared effect. Further, the far-infrared rays can be prevented from being isolated or weaken by the impermeable sheet (13) and the absorbent layer (11).

Even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

What is claimed is:

1. A sanitary napkin with far-infrared radiation comprising:
a napkin body having a plurality of ceramic powders disposed in the napkin body.

2. The sanitary napkin as claimed in claim 1, wherein
the napkin body includes
an absorbent layer having a top and a bottom;
a contact layer mounted on the top of the absorbent layer; and
an impermeable sheet mounted on the bottom of the absorbent layer; and
the plurality of ceramic powders is disposed in the contact layer.

3. The sanitary napkin as claimed in claim 1, wherein
the napkin body includes
an absorbent layer having a top and a bottom;
a contact layer mounted on the top of the absorbent layer;
an impermeable sheet mounted on the bottom of the absorbent layer; and
a surface fiber layer laid on the top of the contact layer; and
the plurality of ceramic powders is disposed in the surface fiber layer.

4. The sanitary napkin as claimed in claim 3, wherein the surface fiber layer is an integral piece.

5. The sanitary napkin as claimed in claim 3, wherein
the absorbent layer further has an anti-leaking groove being looped and formed in the top of the absorbent layer to divide the top of the absorbent layer into a central section and an outer section around the central section; and
the surface fiber layer includes two separate parts respectively being
a central component laid on the central section on the absorbent layer; and
an outer component laid on the outer section on the absorbent layer around the central component.

6. The sanitary napkin as claimed in claim 1, wherein the plurality of ceramic powders is nanometer-sized.

7. The sanitary napkin as claimed in claim 2, wherein the plurality of ceramic powders is nanometer-sized.

8. The sanitary napkin as claimed in claim 3, wherein the plurality of ceramic powders is nanometer-sized.
